(19) United States
(12) Patent Application Publication
(10) Pub. No.: US 2023/0172467 A1
(43) Pub. Date: Jun. 8, 2023

(54) SYSTEM AND METHOD FOR MONITORING A PLURALITY OF BIO-SIGNALS, AND A GATEWAY DEVICE OPERABLE THEREIN

(71) Applicant: AEVICE HEALTH PTE LTD., Singapore (SG)

(72) Inventors: Aiman BIN IBRAHIM, Singapore (SG); Vivian Ci Ai KOH, Singapore (SG); Siti Hamidah BINTE ABDUL HAMID, Singapore (SG); Xiao Rex TAN, Singapore (SG); Yi Yang ANG, Singapore (SG); Wee SER, Singapore (SG)

(21) Appl. No.: 17/998,951

(22) PCT Filed: May 17, 2021
(86) PCT No.: PCT/SG2021/050269

§ 371 Date: Nov. 16, 2022

(30) Foreign Application Priority Data
May 18, 2020 (SG) 10202004626V

(51) Int. Cl. A61B 5/024; A61B 5/00

(52) U.S. Cl. CPC A61B 5/02438; A61B 5/7203; A61B 5/0816

(57) ABSTRACT

According to embodiments of the present invention, a gateway device operable in a system for monitoring a plurality of bio-signals is provided. The gateway device includes a receiver unit configured to receive data and reconstruct the received data to obtain a digital sound signal representative of the plurality of bio-signals; a processing unit configured to determine at least one biometric from the digital sound signal and form a digital signal representative of the plurality of bio-signals and the determined at least one biometric, and to reconstruct the digital signal into two or more data segments, wherein each data segment includes a portion of the digital signal; and a transmitter unit configured to transmit information including the two or more data segments to an external processing module for further processing. According to further embodiments, a system and a method for monitoring a plurality of bio-signals are also provided.

### CROSS-REFERENCE TO RELATED APPLICATION

This application claims the benefit of priority of Singapore patent application No. 10202004626V, filed on 18 May 2020, the content of it being hereby incorporated by reference in its entirety for all purposes.

### TECHNICAL FIELD

Various embodiments relate to a gateway device operable in a system for monitoring a plurality of bio-signals, a system and a method for monitoring a plurality of bio-signals, more specifically, for remote monitoring of at least one person's cardiac, respiratory, or cardiac-respiratory acoustical signal (e.g., physiological signal for brevity).

### BACKGROUND

As technology permeates the fields of medicine, sprouting new standards of care aided by advanced innovations, healthcare for chronic respiratory disease is in need of technology refresh. New technologies are simply not available to better serve the needs of patients suffering from various respiratory ailments such as Asthma and Chronic Obstructive Respiratory Disease (COPD). Technological innovations are rare in this space. Medical devices advancements largely focused on the tracking of medication intake that promotes adherence of patients. Such technologies provide useful but limited insights to caretakers and healthcare professionals. Treatment adherence is a serious challenge in managing chronic respiratory diseases, but it is only part of a bigger problem. The management of chronic diseases due to its nature, requires objective inputs from a wider perspective including societal and governmental intervention and diligent self-monitoring of condition by the sufferer.

The monitoring of symptoms is required before, during and after diagnosis of such diseases. For example, in asthma care, clinicians rely on indications including symptoms of wheeze, cough, breathlessness and chest tightness that vary over time before and during the clinical consultation to make an informed diagnosis. Post diagnosis, the symptom evolutions exhibited by the sufferer are critical information both for the management of the disease and feedback for the treatment plan. In the current state, constant monitoring of such conditions is carried out by sufferers with the help of symptom logbooks in supplement of periodic consultations with their clinical specialist. The consultants may take reference to the symptom logs and verbal interviews with the patient to make clinical decisions in order to prescribe the next appropriate course of action. This process is plagued with adherence issues and logbook entries and verbal interviews are often unreliable due to the subjective nature of such information.

Aside from monitoring conditions for the purpose of collating objective insights, there exists a need for early detection of exacerbation or attacks. For diseases such as Asthma and COPD, acute exacerbation or attacks can lead to life threatening situations if not detected or recognized early by the sufferer or their caregivers. This is especially true if the sufferer is of tender or elder age during time absent from caregiver supervision such as at night during sleep.

A publication, US20160100817A1, discloses a system and method for capturing and outputting data regarding a bodily characteristic. A part of the system performs acoustic acquisition of physiological sound. However, this part is non-wearable.

Other publications, US20170071506A1 and WO2019241674 discuss processing of acoustic signals where undesirable respiratory events are monitored or detected for the processing which requires comparison with either a user's baseline or a known audio/motion data criteria.

Thus, there is a need for a system and method that address at least the problems mentioned herein and above by adopting emerging Internet-of-Things (IoT) technologies to the system and method to at least record or log observations, improve adherence to medications; constantly monitor respiratory health of wearers, enable recognition of occurrence of critical signs of exacerbation or attacks such as occurrences of cough and wheezing on top of constant monitoring of heart and respiratory rate. With the assistance of such a system and method, not only the risk of poor disease management may be averted, objective logs by the system and method can help clinicians better treat the diseases.

### SUMMARY

According to an embodiment, a gateway device in a system for monitoring a plurality of bio-signals is provided. The gateway device may include a receiver unit configured to receive data and reconstruct the received data to obtain a digital sound signal representative of the plurality of bio-signals; a processing unit configured to determine at least one biometric from the digital sound signal and form a digital signal representative of the plurality of bio-signals and the determined at least one biometric, wherein the processing unit is further configured to reconstruct the digital signal into two or more data segments, and wherein each of the two or more data segments comprises a portion of the digital signal, the portion being representative of at least one of: at least one of the plurality of bio-signals, or the determined at least one biometric; and a transmitter unit configured to transmit information including the two or more data segments to an external processing module for further processing.

According to an embodiment, a system for monitoring a plurality of bio-signals is provided. The system may include a wearable acoustic device configured to detect analogue sound signals representative of the plurality of bio-signals, and convert the detected analogue sound signals into data representative of the plurality of bio-signals; a gateway device configured to receive the data from the wearable acoustic device, reconstruct the received data to obtain a digital sound signal, determine at least one biometric from the digital sound signal and form a digital signal representative of the plurality of bio-signals and the determined at least one biometric, reconstruct the digital signal into two or more data segments, and transmit information including the two or more data segments, wherein each of the two or more data segments comprises a portion of the digital signal, the portion being representative of at least one of: at least one of the plurality of bio-signals, or the determined at least one biometric; and a processing module configured to receive the information, and process the received information for visual display and/or audio display to monitor the plurality of bio-signals and/or the determined at least one biometric.

According to an embodiment, a method for monitoring a plurality of bio-signals is provided. The method may include detecting analogue sound signals representative of the plurality of bio-signals; converting the detected analogue sound signals to data representative of the plurality of bio-signals; reconstructing the data to obtain a digital sound signal, determining at least one biometric from the digital sound signal and forming a digital signal representative of the plurality of bio-signals and the determined at least one biometric, reconstructing the digital signal into two or more data segments, wherein each of the two or more data segments comprises a portion of the digital signal, the portion being representative of at least one of: at least one of the plurality of bio-signals, or the determined at least one biometric; and processing information including the two or more data segments for visual display and/or audio display to monitor the plurality of bio-signals and/or the determined at least one biometric.

The invention is defined in the independent claim(s). Further embodiments of the invention are defined in the dependent claims.

### BRIEF DESCRIPTION OF THE DRAWINGS

In the drawings, like reference characters generally refer to like parts throughout the different views. The drawings are not necessarily to scale, emphasis instead generally being placed upon illustrating the principles of the invention. In the following description, various embodiments of the invention are described with reference to the following drawings, in which:

FIG. 1(a) shows a schematic cross-sectional view of a gateway device operable in a system for monitoring a plurality of bio-signals, according to various embodiments.

FIG. 1(b) shows a schematic cross-sectional view of a system for monitoring a plurality of bio-signals, according to various embodiments.

FIG. 1(c) shows a flow chart illustrating a method monitoring a plurality of bio-signals, according to various embodiments.

FIG. 2 shows a schematic diagram depicting an overview of a system for monitoring a plurality of bio-signals, according to various embodiments.

FIG. 3 shows a schematic diagram depicting an exemplary user interface layout on a user interfacing device, according to one embodiment.

FIG. 4(a) shows a schematic diagram illustrating an exemplary wearable acoustic device (or sensor), according to one embodiment.

FIG. 4(b) shows a flow diagram illustrating the circuitry of the exemplary wearable acoustic device of FIG. 4(a).

FIG. 4(c) depicts a packet arrangement of an acquired 3-second acoustic measurement, according to one embodiment.

FIG. 5(a) shows a schematic diagram illustrating an exemplary smart gateway, according to one embodiment.

FIG. 5(b) shows a flow diagram illustrating the circuitry of the exemplary smart gateway of FIG. 5(a).

FIG. 5(c) shows a schematic diagram illustrating a first framework using a model-based framework for remote monitoring of at least one person's bio-signals, according to various embodiments.

FIG. 5(d) shows a schematic diagram illustrating a schema-based framework to be used in tandem with the model-based framework of FIG. 5(c) to form a second framework, according to various embodiments.

FIG. 5(e) shows a schematic diagram illustrating a teacher-based framework to be used in tandem with the model-based framework of FIG. 5(c) to form a third framework, according to various embodiments.

FIG. 5(f) shows a schematic diagram illustrating on-the-air firmware updates used by the teacher-based framework of FIG. 5(e).

FIG. 6 shows a block diagram of a cloud-based platform, according to various embodiments.

FIG. 7 shows a flow chart illustrating a summary of the steps of respiratory rate estimation algorithm, according to various embodiments.

FIG. 8 shows a flow chart illustrating a summary of the steps of abnormal respiratory sounds detection algorithm, according to various embodiments.

FIG. 9 shows a flow chart illustrating a summary of the steps of heart rate detection algorithm, according to various embodiments.

FIGS. 10(a) to 10(e) show examples illustrating how data may be segmented or reconstructed into different data segments, according to one embodiment.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

Embodiments described in the context of one of the methods or devices are analogously valid for the other methods or devices. Similarly, embodiments described in the context of a method are analogously valid for a device, and vice versa.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

In the context of various embodiments, the articles “a”, “an” and “the” as used with regard to a feature or element include a reference to one or more of the features or elements.

In the context of various embodiments, the term “about” as applied to a numeric value encompasses the exact value and a reasonable variance.

As used herein, the term “and/or” includes any and all combinations of one or more of the associated listed items.

As used herein, the phrase of the form of “at least one of A or B” may include A or B or both A and B. Correspondingly, the phrase of the form of “at least one of A or B or C”, or including further listed items, may include any and all combinations of one or more of the associated listed items.

Various embodiments may provide a four-part internet of things (IoT) system developed for physiological vital signs and symptoms surveillance. The system may allow an alert to be pushed to caregivers, family members, authorities, and so on should abnormalities be detected. In addition to benefits to individuals, longitudinal information collected by the system may potentially transform and raise the standard of care of chronic respiratory illnesses. The system involves the use of a gateway device, which is an edge intelligence smart gatewateway that serves both as a computational hub and linkage between various components in the system.

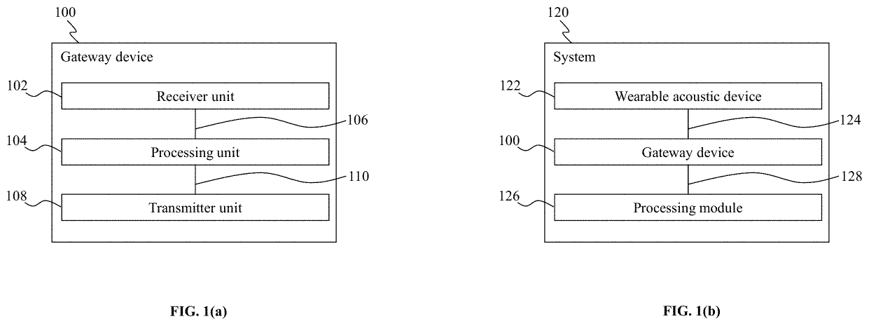

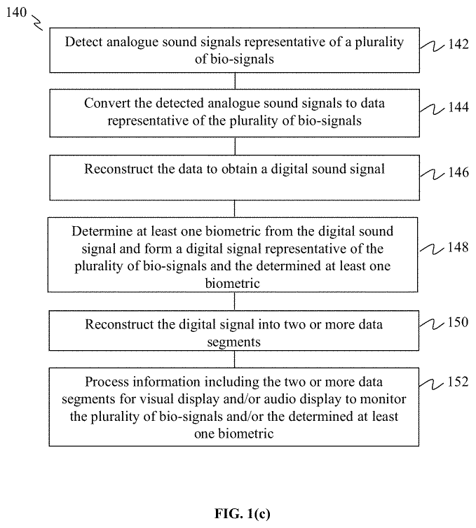

FIG. 1(a) shows a schematic cross-sectional view of a gateway device 100 operable in a system (e.g., 120 of FIG. 1(b)) for monitoring a plurality of bio-signals, according to various embodiments. In FIG. 1(a), the gateway device 100 includes a receiver unit 102 configured to receive data and reconstruct the received data to obtain a digital sound signal representative of the plurality of bio-signals; a processing unit 104 configured to determine at least one biometric from the digital sound signal and form a digital signal representative of the plurality of bio-signals and the determined at least one biometric, wherein the processing unit is further configured to reconstruct the digital signal into two or more data segments; and a transmitter unit 108 configured to transmit information including the two or more data segments to an external processing module for further processing. Each of the two or more data segments may include a portion of the digital signal. The portion may be representative of at least one of: at least one of the plurality of bio-signals, or the determined at least one biometric. In other words, the portion may be representative of at least one of the plurality of bio-signals, or the determined at least one biometric, or any combinations thereof.

The receiver unit 102 and the processing unit 104 are in communication with each other, as depicted by a line 106. The processing unit 104 and the transmitter unit 108 are in communication with each other, as depicted by a line 110.

In the context of various embodiments, the phrase “representative of” may mean indicative of, or including, or at least partially equivalent to.

In various embodiments, each of the two or more data segments may include at least three seconds of data. The two or more data segments may have different data lengths. For example, the two or more data segments may include a first segment and a second segment, wherein the first segment has a data length longer than the second segment.

The first segment may be representative of at least one of: the at least one of the plurality of bio-signals having an occurrence period between about 3 seconds to about 15 seconds, or the determined at least one biometric having an occurrence period between about 3 seconds to about 15 seconds. The second segment may be representative of at least one of: the at least one of the plurality of bio-signals having an occurrence period between about 3 seconds to about 5 seconds, or the determined at least one biometric having an occurrence period between about 3 seconds to about 5 seconds. For example, the first segment and the second segment respectively may include 15 seconds of data and 5 seconds of data; or 10 seconds of data and 3 seconds of data; or 12 seconds of data and 4 seconds of data.

In various embodiments, the plurality of bio-signals may include signals derived from bodily sounds, e.g., respiratory signals, or cardiac signals, or other signals derived from bodily sounds, or any combinations thereof. For example, a respiratory signal may be a normal breath sound or an abnormal breath sound, while a cardiac signal may be a heart sound. The term “respiratory” is meant to also include any abnormal respiratory breathing sounds emitting from a person's chest cavity, for example wheeze, crackles and stridor. Generally, a bio-signal may be a raw physiological signal that may be monitored.

The determined at least one biometric may be selected from the group consisting of a heart rate (HR), a heart rate variability (HRV), a respiratory rate (RR), a respiratory rate variability (RRV) and any combination thereof. In other words, the plurality of bio-signals may be used to detect abnormal breath sounds, estimate HR, HRV, RR, RRV and any combination thereof.

Heart rate described herein refers to the number of beats per minute, while heart rate variability described herein refers to changes in heart rate, with assessment period ranges from very-short-term (beat-to-beat) to short-term (within 24 hours in a day) and also to long term (months or years).

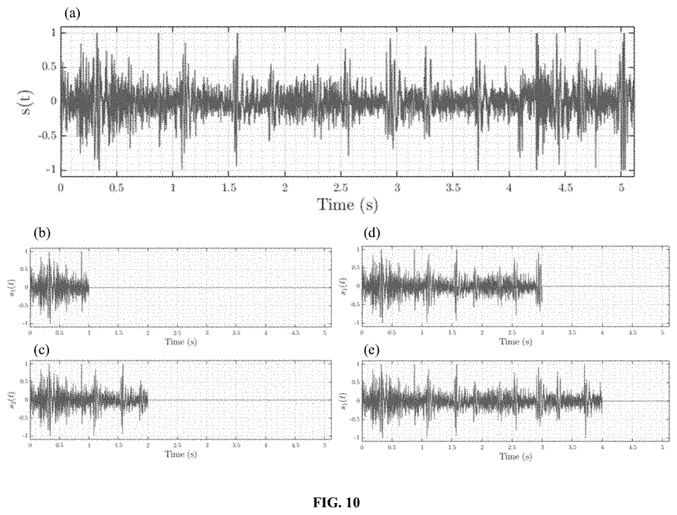

In other words, the gateway device 100 may receive and reconstruct the data to obtain the digital sound signal that may be stored in 16-bit resolution or more. The digital sound signal may also be partially processed and stored as entropy values (for example, 15 seconds worth of entropy values). The gateway device 100 may be adapted to segment the digital signal into a first data segment and a second data segment, with each array including at least 3-second of data. For example, the data may be segmented into lengths of 15 seconds and 5 seconds, or 10 seconds and 3 seconds, or 12 seconds and 4 seconds, respectively, and so on. Respiration occurs with a longer cycle, hence a long segment may be needed to be used to observe it. On the other hand, heart signals may be observed at short intervals. Moreover, abnormal lung sound usually occurs across a short time period and hence may be observed from both long and short segments. FIGS. 10(a) to 10(e) show examples illustrating how data may be segmented or reconstructed into the different data segments. In FIG. 10(a), a full digitized signal s(t) runs from time 0 second to about 5 seconds, the y-axis of the graph in FIG. 10(a) are arbitrary values for amplitude. In an exemplary first data segment of FIG. 10(b), the signal is part of the full digitized signal s(t) and runs from time 0 second to about 1 second. In an exemplary second data segment of FIG. 10(c), the signal is part of the full digitized signal s(t) and runs from time 0 second to about 2 seconds. In an exemplary third data segment of FIG. 10(d), the signal is part of the full digitized signal s(t) and runs from time 0 second to about 3 seconds. In an exemplary fourth data segment of FIG. 10(e), the signal is part of the full digitized signal s(t) and runs from time 0 second to about 4 seconds. The different reconstruction/segmentation may help to focus on the type of biometrics that the system may calculate. For example, FIG. 10(b) has interference from the breath sound because the frame may be too short for the system to identify a breath. Effectively, using additional data segments increases the confidence level of a specific biometric estimation. It should be appreciated that segmentation or reconstruction into data segments is not limited to the examples shown in FIGS. 10(a) to 10(e). Different variations in terms of the lengths of period of the data segment, the start time of the data segment with respect to the start time of the full digitized signal (e.g., not starting at time 0 second as shown in FIGS. 10(b) to 10(e)), amongst others may be possible.

In one embodiment, the receiving unit 102 may include an input port for wired communication with an external wearable acoustic device (not shown in FIG. 1(a)).

In other various embodiments, the receiver unit 102 may be configured to receive the data via a wireless communication protocol for an optimal communication distance ranging up to about 10 m, or the BLE 5 radius of influence (as an example). For example, such a wireless communication protocol may include Bluetooth Low Energy, BLE, which generally allows exchange of data between fixed and mobile devices over short distances using 2.4 GHz radio frequencies.

Alternatively, the receiver unit 102 may be configured to receive the data via a wireless low-energy communication protocol utilizing Low Complexity Communications Codec. This wireless low-energy communication may be in the form of LE Audio, which is based on the enhancement of the Bluetooth Core Specification to enable delivery of audio over BLE protocol, and the inclusion of a new LE Isochronous Channels feature. LE Audio generally consumes less energy than other BLE protocol due to utilization of the Low Complexity Communications Codec, LC3. For example, reference may be made to https://www.anandtech.com/show/15349/bluetooth-sig-announces-le-audio-standard-new-baseline-for-next-decade. In general, LE audio utilizes a specific profile setting under the BLE framework to achieve desirable outcomes and properties.

In various embodiments, the transmitter unit 108 may be configured to transmit the information to the external processing module in an external server or a cloud-based platform (not shown in FIG. 1(a)).

The gateway device 100 may further include a microphone configured to detect ambient sound surrounding the gateway device 100, and an analogue-to-digital converter configured to convert the ambient sound to a digital equivalent. The processing unit 104 may be further configured to remove noise from the digital sound signal based on the digital equivalent of the ambient sound.

In other words, the gateway device 100 may be further adapted to detect ambient sound and remove noise. Desired sound signals are known frequencies or known acoustical patterns of lung sounds. All other frequencies or acoustical patterns may be considered noise, and therefore removed through hardware setup, e.g., analogue and digital filtering; firmware algorithms such as adaptive and statistical signal processing, or both.

The gateway device 100 may further include a filtering circuit and signal amplifier for processing the ambient sound. The gateway device 100 may also include at least one port, e.g., micro-USB, that is adapted for receiving electrical power to drive the electrical components of the gateway device 100.

FIG. 1(b) shows a schematic cross-sectional view of a system 120 for monitoring a plurality of bio-signals, according to various embodiments. In FIG. 1(b), the system 120 includes a wearable acoustic device 122 configured to detect analogue sound signals representative of the plurality of bio-signals, and convert the detected analogue sound signals into data representative of the plurality of bio-signals; a gateway device 100 (FIG. 1(a)) configured to receive the data from the wearable acoustic device 122, reconstruct the received data to obtain a digital sound signal, determine at least one biometric from the digital sound signal and form a digital signal representative of the plurality of bio-signals and the determined at least one biometric, reconstruct the digital signal into two or more data segments, and transmit information including the two or more data segments; and a processing module 126 configured to receive the information, and process the received information for visual display and/or audio display to monitor the plurality of bio-signals and/or the determined at least one biometric. Each of the two or more data segments may include a portion of the digital signal, the portion being representative of at least one of: at least one of the plurality of bio-signals, or the determined at least one biometric.

The wearable acoustic device 122 and the gateway device 100 are in communication with each other, via a wired connection or wirelessly, as depicted by a line 124. The gateway device 100 and the processing module 126 are in communication with each other wirelessly, as depicted by a line 128.

In various embodiments, the system 120 may further include a user interfacing device configured to display the processed information.

In other words, the system 120 may be for monitoring signals derived from bodily sounds, e.g., cardiac-respiratory acoustical signals including heart and respiratory rates; heart and respiratory rate variabilities; and abnormal lung sounds such as cough, wheeze, stridor and crackle in at least a person. The system 120 may include (a) at least one wearable acoustic device 122 for acquiring analogue sound signals from a chest cavity of the person, the wearable acoustic device 122 including an acoustic sensor for capturing acoustical physiological signals; (b) a gateway device 100 for receiving the captured acoustical physiological signals in digital form from the at least one wearable acoustic device 122, the gateway device 100 adapted to extract at least one feature of interest that represents the person's cardiac-respiratory acoustical signal including respiratory and/or heart rate; and (c) a user interfacing device for providing information associated with the at least one feature of interest, wherein the at least one feature of interest is selected from the group consisting of abnormal respiratory sounds, heart rate, heart rate variability, respiratory rate and respiratory rate variability with the optional capability of recording any observation of interest, e.g., the person (user)'s weight, height, or even intake of medication.

The gateway device 100 (described in FIG. 1(b)) may include the same or like elements or components as those of the gateway device 100 of FIG. 1(a), and as such, the same numerals are assigned and the like elements may be as described in the context of the gateway device 100 of FIG. 1(a), and therefore the corresponding descriptions are omitted here.

The wearable acoustic device 122 may include at least one sensing unit configured to detect the analogue sound signals; a converter configured to convert the detected analogue sound signals into the data; and a transmitter configured to transmit the data to the gateway device 100.

For example, the at least one sensing unit may include a MEMS microphone, an optical fiber, or any other acoustic sensing device. The at least one sensing unit may select an operating frequency range closest to the desired frequency. By doing so, the at least one sensing unit may be prevented from picking up significantly low end or significantly high-end noise.

In various embodiments, the converter may be configured by defining different sampling rates. According to Nyquist sampling theorem, frequencies higher than half the sampling rate are naturally removed and do not contribute coherently to the acquired signal.

The wearable acoustic device 122 may further include a conditioning and filtering analogue circuit for removing noise in the analogue sound signals. The conditioning and filtering analogue circuit may include an analogue band pass filter circuit with amplification limit. This is a family of circuit design with wide variation. The tuning parameters may include how high the amplitude cut-off may be and what frequency range the interested signals may mostly reside. For example, a low amplitude cut off and frequency range of about 60 Hz to 200 Hz may be set for the wearable acoustic device 122 to be used to acquire only heart sounds, since heart sounds are relatively loud as compared to other bodily sounds from the chest and sound is of low frequency.

The wearable acoustic device 122 may also include an amplifier configured to amplify the analogue sound signals prior to conversion into the data. Amplification limit may form another filtering function. As lung or heart sound are unlikely to be much louder than the environment loud noises such as slammed doors or loud music, the limit imposed by the hardware for amplification removed the influence of these noises as they may be amplified past the upper limit of the amplitude range, hence losing features. For example, in amplifying a wearer's physiological acoustic signals, a small acoustic sensor condenser microphone of the wearable acoustic device 122 may acquire significantly faint electric signals by varying its capacitance reacting to impeding sound waves. As such, these electric signals may need to be amplified to a value suitable for sampling with an ADC (e.g., the converter). The electric signals produced by the wearable acoustic device 122, which may include a condenser microphone, a micro-electromechanical systems (MEMS) microphone, or an optical acoustic sensor), may be amplified according to a reference voltage level set on the ADC, e.g., 0V-3.3V range. This is not a strict rule in this regard, and hence in other embodiments, the reference voltage level may be 0V to 5V range, or even include inverted voltages, e.g., −3.3V to +3.3V range.

In various embodiments, the wearable acoustic device 122 may be adapted to reconstruct the data into a form of a plurality of data trenches prior to transmission to the gateway device 100. Each of the plurality of data trenches may be a 0.5-second trench.

For example, the wearable acoustic device 122 may be adapted to segment the data into short trenches (e.g., 0.5 or 5 seconds trenches) and transmit the segmented data trenches to the gateway device 100 via a BLE protocol. Other short range wireless protocols such as Zigbee, thread, ANT, other proprietary protocols, and so on may alternatively be used. The wearable acoustic device 122 may also be designed as a narrowband-IoT (NB-IoT) device. The data is sent from a microcontroller (where the sound signal may be sampled) to a BLE module through at least one communication protocol, e.g., serial peripheral interface (SPI); inter-integrated circuit (I2C); or Universal Asynchronous Receiver/Transmitter (UART) communication protocol, which has an 8-bit payload per data packet limitation. Therefore, sampled data at 16-bit resolution or more may need to be fragmented according to the communication protocol before sending through BLE and, then re-combined or reconstructed again.

In various embodiments, the wearable acoustic device 122 may not store 5 seconds of data. The wearable acoustic device 122 may sample data continuously until it gets 0.5 seconds worth of data (due to memory limitations of small processors), sends the data through BLE to the gateway device 100 at a baud rate of 230400, while sampling the next batch. With a baud rate of 230400 or more, the 0.5 seconds width of data may be able to be completely sent, have the microcontroller rest for a bit, before completing the sampling for the next batch of 0.5 seconds worth of data. Only at a docking station that 5 seconds worth of data may be gathered and stored together.

The wearable acoustic device 122 may further include a transmitter for wirelessly transmitting the acquired sound to the gateway device 100.

In various embodiments, the processing module 126 may be provided in an external server or a cloud-based platform.

FIG. 1(c) shows a flow chart illustrating a method 140 monitoring a plurality of bio-signals, according to various embodiments. At Step 142, analogue sound signals representative of the plurality of bio-signals may be detected. At Step 144, the detected analogue sound signals may be converted to data representative of the plurality of bio-signals. At Step 146, the data may be reconstructed to obtain a digital sound signal. At Step 148, at least one biometric may be determined from the digital sound signal and a digital signal representative of the plurality of bio-signals and the determined at least one biometric may be formed. At Step 150, the digital signal may be reconstructed into two or more data segments, wherein each of the two or more data segments includes a portion of the digital signal, the portion being representative of at least one of: at least one of the plurality of bio-signals, or the determined at least one biometric. At Step 152, information including the two or more data segments may be processed for visual display and/or audio display to monitor the plurality of bio-signals and/or the determined at least one biometric.

The plurality of bio-signals may include signals derived from bodily sounds, e.g., respiratory signals, or cardiac signals, or other signals derived from bodily sounds, or any combinations thereof. The determined at least one biometric may be selected from the group consisting of a heart rate, a heart rate variability, a respiratory rate, a respiratory rate variability and any combination thereof. The definitions of bio-signals and biometric as described for the system 120 may be applicable here.

In other words, the method 140 may be for monitoring signals derived from bodily sounds, e.g., respiratory breathing and/or heart rate in a person. The method 140 may include (a) acquiring the cardiac-respiratory acoustical signals from a chest cavity of the person; (b) converting the acquired analogue sound signals to data; (c) wirelessly transmitting the data to the gateway device 100 that is adapted to extract at least one feature of interest that represents the person's respiratory and/or heart rate; (d) providing information associated with the at least one feature of interest, wherein the at least one feature of interest is selected from the group consisting of abnormal respiratory sounds, heart rate, heart rate variability and respiratory rate; and (e) providing a learning function that is either user-directed, e.g. improving itself via user on-the-air (OTA) firmware update or processing-device directed, e.g. multilayer perceptron learning that utilize the information from (c) to perform supervised learning.

The method 140 may include the same or like elements or components as those of the gateway device 100 of FIG. 1(a), and/or the system 120 of FIG. 1(b), and as such, the same numerals are assigned and the like elements may be as described in the context of the gateway device 100 of FIG. 1(a) and/or the system 120 of FIG. 1(b), and therefore the corresponding descriptions may be omitted here.

By “reconstructing” the digital signal into the two or more data segments at Step 150, it is meant to include any method of segmenting the digital signal. Segmenting may be carried out by putting the digital signal into variable arrays in lengths of how long these signals represent. For example, 5 seconds of raw data may be put in an array with the length 20000 datapoints if the sampling rate is at 4 kHz.

In various embodiments of the method 140, each of the two or more data segments may include at least three seconds of data. The two or more data segments may have different data lengths. For example, the two or more data segments may include a first segment and a second segment, wherein the first segment has a data length longer than the second segment. The first segment may be representative of at least one of: the at least one of the plurality of bio-signals having an occurrence period between about 3 seconds to about 15 seconds, or the determined at least one biometric having an occurrence period between about 3 seconds to about 15 seconds. The second segment may be representative of at least one of: the at least one of the plurality of bio-signals having an occurrence period between about 3 seconds to about 5 seconds, or the determined at least one biometric having an occurrence period between about 3 seconds to about 5 seconds. In other examples, the first segment and the second segment respectively may include 15 seconds of data and 5 seconds of data; or 10 seconds of data and 3 seconds of data; or 12 seconds of data and 4 seconds of data.

In various embodiments, the steps of reconstructing the data to obtain the digital sound signal at Step 146 and reconstructing the digital signal into the two or more data segments at Step 150 may be carried out by a gateway device 100. The gateway device 100 may be configured to receive the data via a wireless communication protocol for an optimal communication distance ranging up to about 10 m, or the BLE 5 radius of influence (as an example).

In other embodiments, the gateway device 100 may be configured to receive the data via a wireless low-energy communication protocol utilizing Low Complexity Communications Codec.

In various embodiments, the step of determining at least one biometric from the digital sound signal and forming a digital signal representative of the plurality of bio-signals and the determined at least one biometric at Step 148 may be carried out by a gateway device 100.

The method 140 may further include reconstructing the data into a form of a plurality of data trenches for transmission to the gateway device 100. Each of the plurality of data trenches may be a 0.5-second trench.

In various embodiments, the step of processing information including the two or more data segments at Step 152 may be carried out by a processing module (e.g., 126 of FIG. 1(b)) in an external server or a cloud-based platform.

The method 140 may further include removing undesirable noise in the analogue sound signals prior to converting the detected analogue sound signals to the data at Step 144.

The method 140 may also include detecting ambient sound, e.g., surrounding the gateway device 100; converting the ambient sound to a digital equivalent; and removing noise from the digital sound signal based on the digital equivalent of the ambient sound, prior to the step of reconstructing the digital signal to obtain the two or more data segments at Step 150.

In various embodiments, the step of converting the detected analogue sound signals into the data at Step 144 may be based on a sampling rate of at least 1 KHz to obtain the data with resolution of at least 16-bits and above. In one embodiment, the sampling rate may be more than 2 KHz.

The method 140 may further include amplifying the analogue sound signal prior to converting the detected analogue sound signal to the data at Step 144.

While the method described above is illustrated and described as a series of steps or events, it will be appreciated that any ordering of such steps or events are not to be interpreted in a limiting sense. For example, some steps may occur in different orders and/or concurrently with other steps or events apart from those illustrated and/or described herein. In addition, not all illustrated steps may be required to implement one or more aspects or embodiments described herein. Also, one or more of the steps depicted herein may be carried out in one or more separate acts and/or phases.

In order that the present invention may be fully understood and readily put into practical effect, there shall now be described by way of non-limitative examples only preferred embodiments of the present invention, the description being with reference to the accompanying illustrative figures.

Various embodiments may provide for a four-part system made up of a wearable sensor assembly (or device), a gateway device, a server or data-storage platform (e.g., cloud platform) and at least one user interface (e.g., at least one interfacing mobile application, an online dashboard platform, amongst others).

The wearable sensor assembly (or device) may be an on-line acoustic wearable sensor capable of acquiring lung sound from a wearer's or a user's chest continuously for the early detection of respiratory distress. Lung sound may be collected from the user's chest via a microphone sensor embedded in a wearable electronic device (e.g., the wearable acoustic device 122 of FIG. 1(b)). Acquired lung sound may be sampled at specific frequency range, transmitted to and processed in parts in a gateway device (e.g., 100 of FIG. 1(a)) and a cloud platform in order to determine if any acquired lung sound is abnormal. Meaningful features may be extracted from the acquired signals, from which occurrence of abnormalities in respiratory sounds including wheezing, stridor, crackles and cough may be identified, and respiratory and heart rates and their variabilities may be calculated or at least estimated. Relevant information may be pushed to a mobile application accessible by users in real-time, also providing notification in the event where an abnormality is detected. This system may enable early detection of acute exacerbation of respiratory ailments such as asthma and acute bronchitis, enhancing care for chronic sufferers of respiratory diseases. The system may also allow for an optional capability of logging verbal interview, medication adherence, or any observation of interest.

The four-part system may be of an IoT architecture with edge intelligence including four major components in three different nodes. Each node may be dedicated to a specific set of tasks working in cooperation with the others.

The main functions of the system may be as follow:
acquisition of physiological signal from a wearer's chest;
conditioning of physiological signals acquired;
digital signal processing of physiological signals acquired for the extraction of meaningful features;
translation of extracted feature into useful information (translation occur by applying algorithms to the collected sound signals which calculates the desired information such as wheeze occurrence, respiratory rate or heart rate);
visualization of information on user interfaces;
storage of information.

For example, the information may be stored in a remote database within a virtual private cloud (VPC) in an AWS cloud platform.

In an example, the system may be suitable for carrying out a method for monitoring heart and respiratory rates, heart and respiratory rate variabilities, and abnormal lung sounds including cough, wheeze, stridor and crackle in a person.

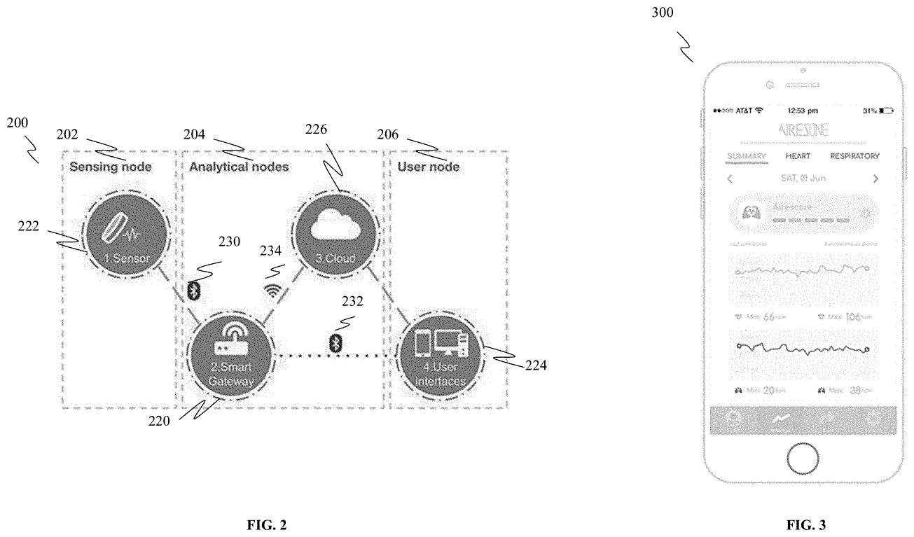

The four-part system 200 may be illustrated by a schematic diagram as shown in FIG. 2. The four-part system may be described in similar context to the system 120 of FIG. 1(b), and hence components of the four-part system may also be respectively described in similar context to the components of the system 120 of FIG. 1(b).

In FIG. 2, The wearable sensor assembly (or device) 222 may be provided at a sensing node 202 that is wirelessly in communication with the smart gateway 220 (or gateway device) provided at analytical nodes 204 e.g., using a BLE protocol 230 or LE Audio. The wearable acoustic device 222 (e.g., wearable acoustic device 122 of FIG. 1(b)) may be a wearable sensor device that is placed or disposed on a person's body, for example, at or near the chest area for acquiring sounds emitting from or near the person's chest area. The wearable acoustic device 222 may carry out certain signal processing as described herein before transmitting the data to the analytical nodes 204.

The smart gateway 220 may communicate with a cloud platform 226, for example, via internet connection 234 at the analytical nodes 204. For example, the cloud platform 226 may include an AWS cloud platform. In other words, the analytical nodes 204 may include the smart gateway 220 and the cloud-based platform AWS server. Both the smart gateway 220 and AWS further processes the digital signals to obtain data associated with the person's heart and respiratory rates, and heart and respiratory rate variabilities, and also to determine whether the person is emitting any abnormal lung sounds such as cough, wheeze, stridor and crackle. Processed information from the cloud platform 226 may be extracted by or transmitted to user interfaces 224 at a user node 206, providing any notifications or alerts in the event where an abnormal heart/respirator sounds may be obtained from the person. The user interfaces 224 may also communicate with the smart gateway 220, e.g., via a BLE protocol 232.

An example of one of the use interfaces 224 may be provided by a user interface layout presented on a user interfacing device 300, as shown in FIG. 3.

In various embodiments, data may be transmitted across the various nodes wirelessly.

The system design forms a complete path from sensing to data visualization, guided by feedback from an international group of about 40 clinicians. It is noteworthy that due to the edge intelligence design, the smart gateway 220 performed most of the computation required and outputs lean data. Hence, it is possible for the smart gateway 220 to be cloud linked via other forms of communication with lower data rates such cellular solutions.

Wearable Acoustic Device

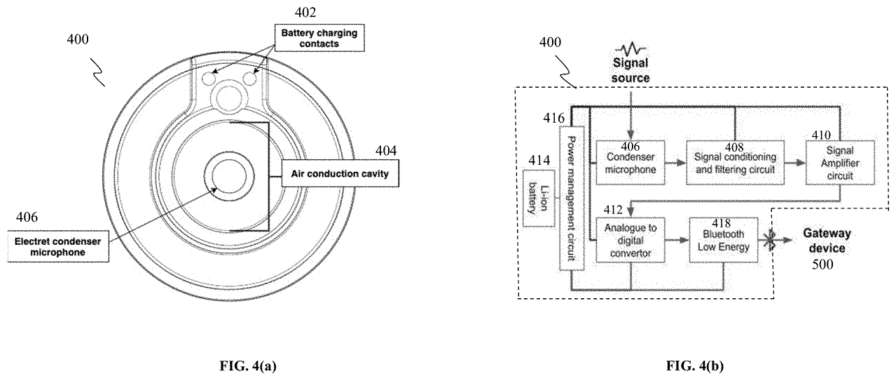

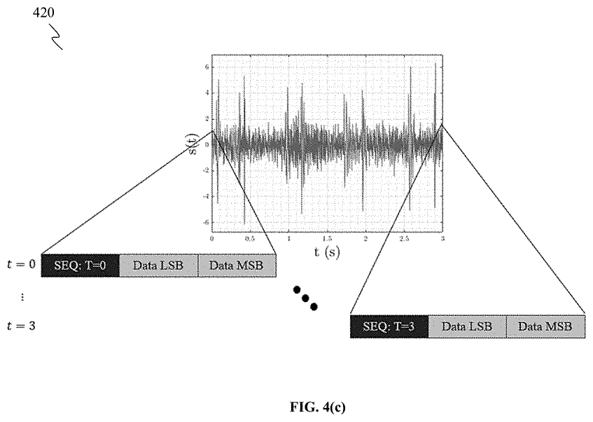

FIG. 4(a) shows a schematic diagram illustrating an exemplary wearable acoustic device (or sensor) 400 (e.g. described in similar context to the acoustic device 222 of FIG. 2). FIG. 4(b) shows a flow diagram illustrating the circuitry of the exemplary wearable acoustic device 400 of FIG. 4(a).

The wearable acoustic device 400 may be an acoustic sensor designed to acquire chest sound from the wearer when worn using a silicone patch coated with medical silicone adhesive. Chest sound emanating from the chest of the wearer may be conducted from the skin of the wearer, in contact with or without a diaphragm, through a hemispheric air conduction bell chamber, for example, providing an air conduction cavity 404 at the bottom of the device 400 to a recessed electret condenser microphone 406 placed at the pole of the hemisphere. Essentially, raw acoustic signal is gathered by the wearable acoustic device 400 with the condenser microphone 406 at the apex of the acoustic cavity dome 404.

The wearable acoustic device 400 may be powered by a rechargeable lithium ion battery pack 414 connectable to the battery charging contacts 402. The battery charging contacts 402 may be linked to a charging circuit disposed within the wearable acoustic device 400 that charges the rechargeable lithium ion battery pack 414. The wearable acoustic device 400 may enable wire-free operations. The wearable acoustic device 400 may include a power management circuit 416 to manage the power supplied to the various components of the wearable acoustic device 400.

Signal from the condenser microphone 406 may be passed through a conditioning and filtering analogue circuit 408 resulting in removal of signals exceeding both frequency and amplitude characteristics of target sound as noise before amplification by a signal amplifier circuit 410. The amplified signal is then converted to digital signal, by an analogue to digital convertor 412, with no less than 2 kHz sampling rate at a resolution of above 16-bits. A minimum of 2 kHz sampling rate is required as the majority of interesting chest sound has a frequency between 50 Hz to 1 kHz. In order to preserve features in the signal, a minimum resolution of 16-bits may be used. According to Nyquist-Shannon Sampling Theorem, the minimum sampling frequency should be at least two times the maximum frequency of interest (in this case 1 kHz) to prevent aliasing and loss of information during digital signal processing.

The acquired signals first pass through an analogue circuit resulting in amplification and with minimal distortion before converting into digital signal at a sampling rate of 4 kHz, hence effectively limiting frequency of signal to a band of 50 Hz (i.e. a lower limit of the electret condenser microphone 406) to 2 kHz (Nyquist frequency) and resolution of at least 16 bits (configurable up to 32 bits). This digitized signal is segmented into 0.5 seconds trenches and transmitted to a gateway device via BLE protocol. Alternatively, 5 seconds or more worth of data may be gathered at the docking station.

Acoustic signal acquired is processed and digital signal may be transmitted to the smart gateway 500 (interchangeably referred to as the gateway device) via Bluetooth, more specially, Bluetooth Low Energy (BLE) 418.

Transmission to the Smart Gateway Via BLE

BLE has a limited data throughput and due to the periodic activation of the BLE radio, it is prone to transmission loss, e.g., data loss and packet loss. Therefore, a robust error detection method is required to ensure the integrity of the data transferred.

Common methods using cyclic-redundancy check (CRC), or error-correcting code (ECC) requires a large bandwidth, i.e., high code-ratio in ECC. However, both CRC and ECC methods do not provide the ability to sequence the data packets.

Transferring data with a resolution more than 8 bits may be critical as 8 bits is the size limit data size per byte packet. This means that data would need to be separated in multiple packets before transfer, and recombined back post transfer. Since the data is separated into multiple packets, the sequence of the data received is important for recombination of data, and any unpredictable data loss would significantly complicate this process. The occurrence of data loss affects the data extraction on a receiver's end, and if data loss is not detected, it would alter the audio signal information, as audio waves are a combination of multiple continuous sine waves.

Therefore, a protocol providing error detection and packet sequencing capability is required.

BLE 5.0 has made a significant leap in terms of reducing the overall power consumption while increasing the data throughput. However, the performance may be compensated by either one of the following specifications.

For example, to enable BLE to be more or most power efficient, baud rate would need to be set to the lowest which is 9600. But data throughput would suffer due to the reduced packets transmission rate. To have the highest data throughput, baud rate would need to be at the highest which may be 930400. But power consumption would in turn suffer due to the high transmission rate of the packets. This correlates to the abovementioned point of having the data bandwidth requirement is to be reduced. Thus, a lower baud rate may be selected to effectively reduce the power consumption.

In other words, to obtain a continuous real-time data acquisition, a successful data reception rate is more or equal to the data transmission rate and the data bandwidth size is limited. Following this, the error detection method ought to have the least effect on the bandwidth size. Most error detection methods have requirements that significantly increase the total data bandwidth. This is due to the need of appending the data with error detection bits. Additionally, most error detection methods do not cofunction as a data sequencer, which is required in this protocol since the data is separated prior to transmission.

This protocol may be utilized by both the wearable acoustic device 400 and the smart gateway 500. After acquiring the acoustic measurements, a microprocessor (MCU) in the wearable acoustic device 400 may prepare the data in the following 24-bit packet for each 16-bit information: an 8-bit sequencer (SEQ), an 8-bit least significant bit (LSB), an 8-bit most significant bit (MSB). The sequencer, which cofunction as an error detection tool on the receiver's end, may be incremented with each data pair from 0 to 255 and then overflow back to zero for the next set. As an example, FIG. 4(c) depicts the packet arrangement of an acquired 3-second acoustic measurement, s(t) 420.

The smart gateway 500 receives the packet streams and reconstructs the acoustic measurements using pattern detection. The pattern may be set during data preparation and extract the data accordingly while mitigating any errors due to data lost. The pattern detection method may be configurable in size. When pattern size is larger, the pattern detection method may work more effectively in terms of extracting the packets of data correctly. A main problem of transmitting the audio data in a waveform is that audio data increment and decrease periodically and hence, there is a high probability that the data itself appears like the sequencer. By utilizing a pattern size that is sufficiently large, the probability of the data detected as the sequencer may be reduced.

In unlimited increment sequencing, Equation 1 below may be used for pattern detection:

$$Pattern detected \to if ( raw \text{?} ⩵ ( raw \text{?} + 1 ) ) \&\& ( raw \text{?} ⩵ ( raw \text{?} + 1 ) ) \&\& ( raw \text{?} ⩵ ( raw \text{?} + 1 ) ) \&\& ( raw \text{?} ⩵ ( raw \text{?} + 1 ) ) \&\& ( raw \text{?} ⩵ ( raw \text{?} + 1 ) ) \&\& ( raw \text{?} ⩵ ( raw \text{?} + 1 ) ) . \qquad Equation 1$$

$$\text{?} \text{indicates text missing or illegible when filed}$$

Based on a test example by applying Equation 1 to pattern sizes of 3, 6, 9, 12, 15 and 19, it was found that the pattern size needed to cleanly extract audio data (representative of bio-signals) from the wearable acoustic device 400 is 19.

$$Pattern size = 3 :$$

$$Pattern detected \to if ( ( raw\_data [ ( index + 18 ) ] \text{?} ) \&\& ( raw\_data [ ( index + \text{?} ) ] \text{?} ) \&\& ( raw\_data [ ( index + \text{?} ) ] \text{?} ) \&\& ( raw\_data [ ( index + \text{?} ) ] \text{?} ) \&\& ( raw\_data [ ( index + \text{?} ) ] \text{?} ) \&\& ( raw\_data [ ( index ) + \text{?} ] \text{?} ) \text{?} ( raw\_data [ ( index ) ] \text{?} ) ) ,$$

$$\text{?} \text{indicates text missing or illegible when filed}$$

| | | | | | | | | | | | | | | | | | | |
|---|---|---|---|---|---|---|---|---|---|---|---|---|---|---|---|---|---|---|
| S1 | [symbol] | [symbol] | S2 | [symbol] | [symbol] | S3 | [symbol] | [symbol] | S4 | [symbol] | [symbol] | S5 | [symbol] | [symbol] | [symbol] | [symbol] | [symbol] | S7 |

| | | | | | | |
|---|---|---|---|---|---|---|
| FA | FB | FC | FD | FE | [symbol] | [symbol] |

[symbol]

indicates data missing or illegible when filed

$$Pattern size = 6 :$$

$$Pattern detected \to if ( ( raw\_data [ ( index + 18 ) ] \text{?} ) \&\& ( raw\_data [ ( index + \text{?} ) ] \text{?} ) \&\& ( raw\_data [ ( index + 12 ) ] \text{?} ) \&\& ( raw\_data [ ( index + \text{?} ) ] \text{?} ) \&\& ( raw\_data [ ( index + \text{?} ) ] \text{?} ) \&\& ( raw\_data [ ( index + \text{?} ) ] \text{?} ) \&\& ( raw\_data [ ( index ) ] \text{?} ) ) ,$$

$$\text{?} \text{indicates text missing or illegible when filed}$$

| | | | | | | | | | | | | | | | | | | |
|---|---|---|---|---|---|---|---|---|---|---|---|---|---|---|---|---|---|---|
| S1 | [symbol] | [symbol] | S2 | [symbol] | [symbol] | S3 | [symbol] | [symbol] | S4 | [symbol] | [symbol] | [symbol] | [symbol] | [symbol] | [symbol] | [symbol] | [symbol] | S7 |

| | | | | | | |
|---|---|---|---|---|---|---|
| FB | FC | FD | FE | [symbol] | [symbol] | [symbol] |

[symbol]

indicates data missing or illegible when filed

$$Pattern size = 9 :$$

$$Pattern detected \to if ( ( raw\_data [ ( index + 18 ) ] \text{?} ) \&\& ( raw\_data [ ( index + \text{?} ) ] \text{?} ) \&\& ( raw\_data [ ( index + 12 ) ] \text{?} ) \&\& ( raw\_data [ ( index + \text{?} ) ] \text{?} ) \&\& ( raw\_data [ ( index + \text{?} ) ] \text{?} ) \&\& ( raw\_data [ ( index + \text{?} ) ] \text{?} ) \&\& ( raw\_data [ ( index ) ] \text{?} ) ) ,$$

$$\text{?} \text{indicates text missing or illegible when filed}$$

| | | | | | | | | | | | | | | | | | | |
|---|---|---|---|---|---|---|---|---|---|---|---|---|---|---|---|---|---|---|
| S1 | [symbol] | [symbol] | S2 | [symbol] | [symbol] | S3 | [symbol] | [symbol] | S4 | [symbol] | [symbol] | [symbol] | [symbol] | [symbol] | [symbol] | [symbol] | [symbol] | S7 |

| | | | | | | |
|---|---|---|---|---|---|---|
| FC | FD | FE | [symbol] | [symbol] | 01 | 02 |

[symbol]

indicates data missing or illegible when filed

$$Pattern size = 12 :$$

$$Pattern detected \to if ( ( raw\_data [ ( index + 18 ) ] \text{?} ) \&\& ( raw\_data [ ( index + \text{?} ) ] \text{?} ) \&\& ( raw\_data [ ( index + 12 ) ] \text{?} ) \&\& ( raw\_data [ ( index + \text{?} ) ] \text{?} ) \&\& ( raw\_data [ ( index + \text{?} ) ] \text{?} ) \&\& ( raw\_data [ ( index + \text{?} ) ] \text{?} ) \&\& ( raw\_data [ ( index ) ] \text{?} ) ) ,$$

$$\text{?} \text{indicates text missing or illegible when filed}$$

| | | | | | | | | | | | | | | | | | | |
|---|---|---|---|---|---|---|---|---|---|---|---|---|---|---|---|---|---|---|
| S1 | [symbol] | [symbol] | S2 | [symbol] | [symbol] | S3 | [symbol] | [symbol] | [symbol] | [symbol] | [symbol] | [symbol] | [symbol] | [symbol] | [symbol] | [symbol] | [symbol] | S7 |

| | | | | | | |
|---|---|---|---|---|---|---|
| FD | FE | [symbol] | [symbol] | 01 | 02 | 03 |

[symbol]

indicates data missing or illegible when filed

$$Pattern size = 15 :$$

$$Pattern detected \to if ( ( raw\_data [ ( index + \text{?} ) ] \text{?} ) \&\& ( raw\_data [ ( index + \text{?} ) ] \text{?} ) \&\& ( raw\_data [ ( index + 12 ) ] \text{?} ) \&\& ( raw\_data [ ( index + \text{?} ) ] \text{?} ) \&\& ( raw\_data [ ( index + \text{?} ) ] \text{?} ) \&\& ( raw\_data [ ( index + \text{?} ) ] \text{?} ) \&\& ( raw\_data [ ( \text{?} ) ] \text{?} ) ) ,$$

$$\text{?} \text{indicates text missing or illegible when filed}$$

| | | | | | | | | | | | | | | | | | | |
|---|---|---|---|---|---|---|---|---|---|---|---|---|---|---|---|---|---|---|
| S1 | [symbol] | [symbol] | S2 | [symbol] | [symbol] | S3 | [symbol] | [symbol] | S4 | [symbol] | [symbol] | [symbol] | [symbol] | [symbol] | [symbol] | [symbol] | [symbol] | S7 |

| | | | | | | |
|---|---|---|---|---|---|---|
| FE | [symbol] | [symbol] | 01 | 02 | 03 | 04 |

[symbol]

indicates data missing or illegible when filed

$$Pattern size = 19 :$$

$$Pattern detected \to if ( ( raw\_data [ ( index + 18 ) ] \text{?} ) \&\& ( raw\_data [ ( index + \text{?} ) ] \text{?} ) \&\& ( raw\_data [ ( index + 12 ) ] \text{?} ) \&\& ( raw\_data [ ( index + 9 ) ] \text{?} ) \&\& ( raw\_data [ ( index + \text{?} ) ] \text{?} ) \&\& ( raw\_data [ ( index + \text{?} ) ] \text{?} ) \&\& ( raw\_data [ ( index ) ] \text{?} ) )$$

$$\text{?} \text{indicates text missing or illegible when filed}$$

| | | | | | | | | | | | | | | | | | | |
|---|---|---|---|---|---|---|---|---|---|---|---|---|---|---|---|---|---|---|
| S1 | [symbol] | [symbol] | S2 | [symbol] | [symbol] | [symbol] | [symbol] | [symbol] | S4 | [symbol] | [symbol] | [symbol] | [symbol] | [symbol] | [symbol] | [symbol] | [symbol] | S7 |

| | | | | | | |
|---|---|---|---|---|---|---|
| [symbol] | [symbol] | 00 | 01 | 02 | 03 | 04 |

[symbol]

indicates data missing or illegible when filed

Transmission to the Smart Gateway Via LE Audio

As discussed above, transmission to the smart gateway 500 via BLE involves data collection, data segmentation and data preparation by the wearable acoustic device 400, while error detection, data recombination, data pre-processing, algorithm calculation and publication of results are performed by the smart gateway 500. Audio transfer therebetween may be based on a proprietary profile. An alternative to transmission to the smart gateway 500 via BLE, LE Audio may be used instead.

For LE Audio, data collection and data preparation are performed by the wearable acoustic device 400, without involving data segmentation. Data pre-processing, algorithm calculation and publication of results are performed by the smart gateway 500, without involving error detection and data recombination. LE Audio transfer therebetween involves data compression (encoding), data transfer, input output verification, packet loss concealment and data decompression (decoding). LE Audio allows for a dedicated audio transfer solution for BLE devices, that is an all-in-one plug and play package. Due to data compression in the LE Audio transfer, there may be possibility of higher audio data information transfer rate and lower power consumption to provide more efficient data transfer as compared to the BLE protocol.

Smart Gateway

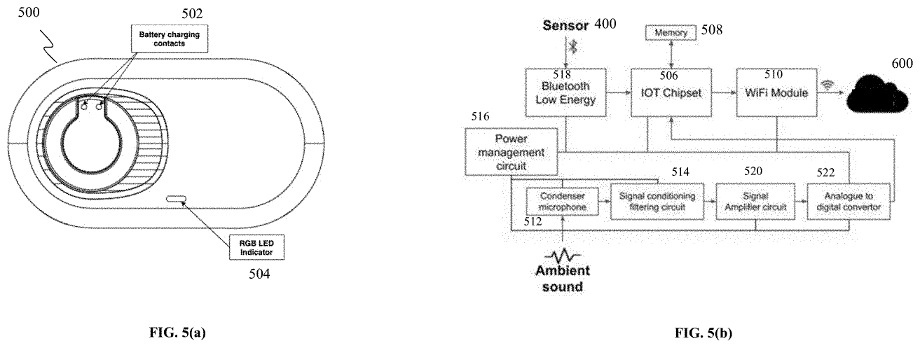

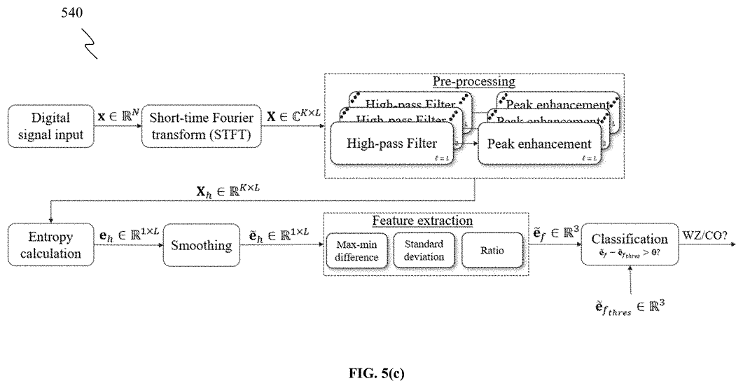

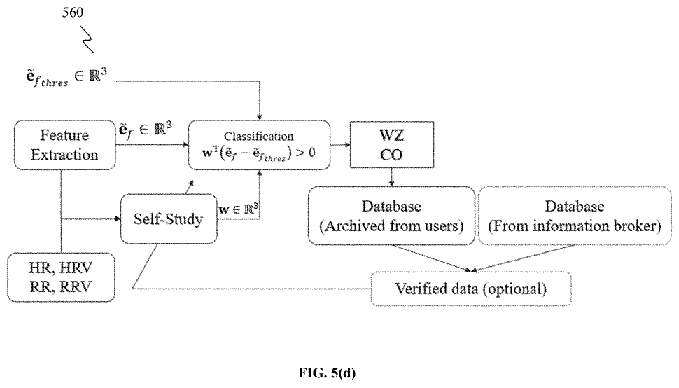

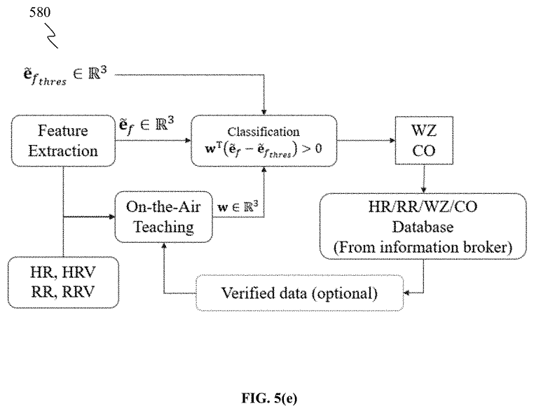

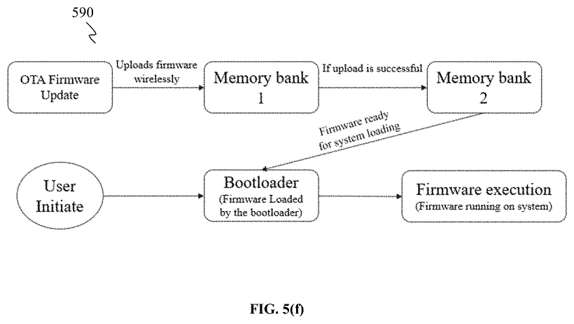

FIG. 5(a) shows a schematic diagram illustrating an exemplary smart gateway 500 (e.g., described in similar context to the gateway device 100 of FIG. 1(a)). FIG. 5(b) shows a flow diagram illustrating the circuitry of the exemplary smart gateway 500 of FIG. 5(a).

The smart gateway 500 is an edge intelligence gateway device that serves both as a computational hub and linkage from the wearable acoustic device 400 to a cloud 600. Digital signal arriving from the wearable acoustic device 400 is segmented into short periods via BLE 518 and processed using a series of digital signal processing algorithms to extract features of interest, i.e., information representative of bio-signals. These algorithms may identify abnormal respiratory sounds, estimates heart rate, heart rate and respiratory rate and variabilities thereof. The smart gateway 500 may be configured to process the digital signals in its entirety resulting in output of useful information directly or processes the digital signals by parts to output relevant features to the cloud 600 where more computationally intensive methods may be implemented. Examples of computationally intensive methods carried out at the cloud 600 are further discussed in the following sections.

As shown in FIGS. 5(a) and 5(b), the smart gateway 500 may indicate charging contacts 502 adapted to receive power to drive the components of the smart gateway 500 through a power management circuit 516, and a red-green-blue (RGB) light emitting diode (LED) indicator 504. The power management circuit 516 of the smart gateway 500 may drive an Internet of Things (IoT) Chipset 506, along with a memory 508 to store e.g., the digital signal processing algorithms and processed information therefrom, and a Wi-Fi module for wirelessly communication with the cloud 600. For example, powered by a 5V DC supply delivered through a micro-USB port at the charging contacts 502, the smart gateway 500 may also include a secondary function of detecting ambient sound for the purpose of environmental sound detection and noise cancelling. In this regard, a condenser microphone 512 may be disposed within the smart gateway 500 for detecting ambient sound nearer or surrounding the smart gateway 500. The ambient sound may be processed via a signal conditioning filtering circuit 514 and a signal amplifier circuit 520. The processed ambient sound may then be converted into a digital equivalent via an analogue to digital converter 522, also disposed in the smart gateway 500. The digital equivalent may be fed to the IoT Chipset 506 for further processing.

The digital signal processing algorithms performed by the smart gateway 500 may be based on three frameworks, as described below, with respect to localizing occurrences of abnormal respiratory sound.

A first framework uses a model-based framework 540 for remote monitoring of at least one person's bio-signals, as shown by a schematic diagram in FIG. 5(c). The model-based framework 540 is based on a set of extracted features and uses a pre-derived model to analyze the bio-signals performance and detect abnormal respiratory sound occurrences. It should be appreciated that the extracted features (shown in FIG. 5(c)) are merely for illustrative purposes and may differ from extracted features in other examples and embodiments described herein. The key concept is that current known features and their temporal evolution are utilized to analyze the performance. In the model-based framework 540, the digital signal input representative of abnormal respiratory sound may be processed using short-time Fourier transform (STFT), filtered through high-pass filter(s) and undergo peak enhancement. Subsequently, entropy calculation, smoothing, feature extraction and classification may be performed. In feature extraction, criteria of maximum-minimum, standard deviation and ratio may be involved.

The model-based framework 540 may be used in tandem with a schema-based framework 560 to form the smart gateway 500's second framework, as shown in FIG. 5(d). The smart gateway 500 continues to create associations between the features and the measured parameters. The objection of a self-study module of the schema-based framework 560 is to determine the appropriate weights, w∈ [symbol] ³ for the features to maximize the classification performance.

For example, let

$$\text{?} {3}^{} \qquad Equation 2$$

$$\text{?} \text{indicates text missing or illegible when filed}$$

where \tilde{e} [symbol] \tilde{e}_{z,999} \tilde{e} [symbol] are the temporal max-min difference, standard deviation, and the ratio of the entropy \tilde{e}ₕ∈ [symbol] ^{1×L} across L temporal frames, respectively. Initially, the smart gateway 500 initializes the weights to ⅓; however, as the smart gateway 500 understands the user, the instantaneous HR, HRV, RR, and RRV may augment the current weight values to emphasize the features that enhance the classification.

The classification may consider the feature extraction as provided in Equation 2 and the weight of w∈ [symbol] ³ to evaluate based on a threshold as shown in Equation 3.

w^{T}(\tilde{e}_{f}−\tilde{e}_{f}_{ₜₕᵣₑₛ})>0 Equation 3

Upon classification, detection of wheeze and/or cough (WZ/CO) may be determined. The classified performance may be stored in a database archived from users and/or from an information broker, which may in turn be optionally used as verified data for the self-studying.

In other examples, the model-based framework 540 may be used in tandem with a teacher-based framework 580 to form the smart gateway 500's third framework, as shown in FIG. 5(e). In the teacher-based approach, the smart gateway 500 performs a student's role and periodically receives insights from a “teacher”, which updates the weight w∈ [symbol] ³ via on-the-air firmware updates 590, as shown in FIG. 5(f).

The main differences between the teacher-based framework 580 and the schema-based framework 560 lie in that the teacher-based framework 580 uses the on-the-air firmware updates 590 instead of the self-studying module, and only database from information broker is used. The on-the-air firmware updates 590 may involve the uploading of the firmware wirelessly to a memory bank 1 and if successfully, the firmware is further uploaded to a memory bank 2. When the firmware is ready for system loading, the firmware may be loaded by a bootloader that may be initiated by a user. The firmware may then be executed by running the firmware on the smart gateway 500 (i.e. system as indicated in FIG. 5(f)).

The Cloud Platform

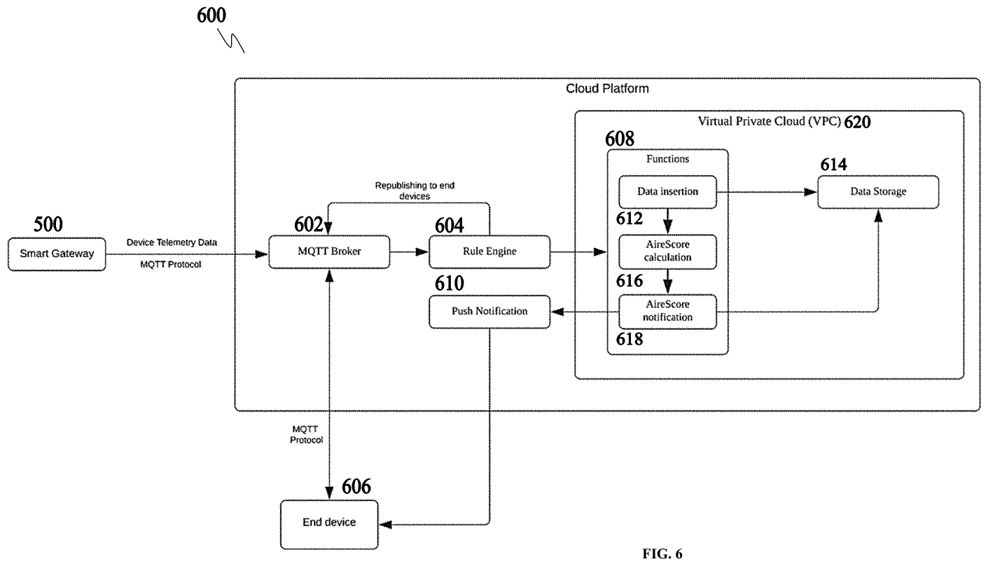

A general sign flow and evolution of a cloud platform, e.g., a AWS cloud platform, is described as shown in FIG. 6.

The gateway device (e.g. the smart gateway 500) connects and publishes device telemetry data to MQTT Broker 602 over secure Message Queuing Telemetry Transport (MQTT). Data received from the MQTT Broker are forwarded to the Rules Engine 604. The Rule Engine 604 may run a set of rules on the input data and if any condition matches, then corresponding actions may be executed by the Rule Engine 604. Device telemetry data published from a predetermined topic may be republished to the end device (at 606) and forwarded to a series of processing steps (at 608).

Functions within the processing steps perform the following actions.
Data insertion (at 612): Device telemetry data are inserted into the database 614 for storage, allowing for long-term trend monitoring by end users.
AireScore calculation (at 616): This function calculates the vital information from features of interest received from the smart gateway 500 and then calculates the AireScore. AireScore is a customisable aggregate score to rate wellness of the wearer. It takes into consideration the variability of heart and respiratory rates and occurrence of abnormal lung sounds in the past X number of hours while the user is wearing the wearable acoustic device 400. The number of hours to consider may be a user input or as recommended by the doctors. It is a summary score that gives an indication if the wearer is well. These vital information are the meta data from the algorithms. For example, in order to detect an episode of wheeze or crackles, these features may be the entropy features of the lung sound
AireScore notification (at 618): This function assesses if a notification should be pushed to the user. If the score falls below a certain range, a push notification 610 is sent to a mobile app. A new notification record is inserted into the database 614 to store this notification information. Airescore is a 0 to 100% nature of score with 0 being very poor and 100% being normal. It may also be represented in steps of 20% as exemplified in one embodiment which airescore is 1 to 5 which each step represents 20%. The threshold again may be a user input or set by a clinician. However, in general, a score of lower than 60% is considered abnormal as it indicated that abnormal lung sounds were detected in the past X number of hours.

The functions 608 and the data storage 614 may be disposed in a virtual private cloud (VPC) 620. The functions 608 may be triggered by interaction on the mobile app.

AireScore

As explained above, the AireScore is an indicative score that may be configured and reconfigured to indicate the performance of different specific sets of wellness and vital signs. In general, AireScore presents to users at-a-glance wellness information of the wearer. The score is a summation of contributions from various matrices measurable using one or more of AireSone devices or/and partner devices.

AireSone devices are capable of measuring the following wellness and vital signs of a wearer and as such the AireScore is computed using these matrices, as follow, as input:
Heart rate (HR)
Heart rate variability (HRV)
Respiratory rate (RR)
Respiratory rate variability (RRV)
Occurrence of Abnormal lung sounds (ABS)
Occurrence of cough (CGH)

Additional matrices may include measurement of motion and sleep state.

AireScore is a 0 to 100 discrete state scoring system that may be segmented into a bulk scoring score (example: 1 to 5, where 5 represents optimal wellness state). The current mathematical model to compute AireSone in general use is as follows. The computation of airescore falls under one of the following computation methods depending on the state of use and intended purpose of device use.

There are three cases under which Airescore is calculated differently.
A. New user with no user input
B. New user with user input parameters
C. Existing users with no user input who have accumulated at least 30 hours of use over 7 separate days.

Examples of default computation are shown below.

```
For a 1 to 5 scare Airescore, each score represents 20%.
If
  any matrix is out of range in latest measurement
    AireScore == 1
else if
  any matrices is out of range for more than x measurements in the
past 240 measurements
```

| | |
|---|---|
| for 0< x =< 2 | ; AireScore == 3 |
| for 3=< x < 8 | ; AireScore == 2 |
| for x >= 8 | ; AireScore == 1 |

```
else if
  any matrix is out of range for at least 1 but not more than 4
measurements in the past 2400 measurements
    AireScore == 4
else if
  any matrix is out of range for more than 4 measurements in the
past 2400 measurements
    AireScore == 3
else
    AireScore == 5
Case 1: Normal range for matrices for new user without user input:
Heart rate
^{#}hr_{low} < HR < ^{#}hr_{high}
Heart rate variability
HRV < ^{#}hrv_{high}
Respiratory rate
^{#}rr_{low} < RR < ^{#}rr_{high}
Respiratory rate variability
RRV < ^{#}rrv_{high}
Abnormal Lung Sound (ABS) & Cough (CGH)
ABS > 0
CGH > 3
Case 2: Normal range for matrices for new user with user input
Heart rate
⁺hr_{low} < HR < ⁺hr_{high} ;
Given that: Range (⁺hr_{low}, ⁺hr_{high}) < A×Range (^{#}hr_{low}, ^{#}hr_{high})
where A is the scaling factor of HR & HRV with a value range of 1 to
1.4.
Heart rate variability
HRV < ⁺hrv_{high}
Given that: ⁺hrv_{high} < (1/A)×^{#}hrv_{high})
where A is the scaling factor of HR & HRV with a value range of 1 to
1.4.
Respiratory rate
⁺rr_{low} < RR < ⁺rr_{high}
Given that: Range (⁺rr_{low}, ⁺rr_{high}) < B×Range (^{#}rr_{low}, ^{#}rr_{high})
where B is the scaling factor of RR and RRV with a value range of 1 to
1.2.
Respiratory rate variability
RRV < ⁺rrv_{high}
Given that: ⁺rrv_{high} < (1/B)×^{#}rrv_{high})
where B is the scaling factor of RR & RRV with a value range of 1 to
1.2.
Abnormal Lung Sound (ABS) & Cough (CGH)
ABS > 0
CGH > 3
Case 3: Normal range for matrices for existing without user input
Heart rate
(0.4× ^{avg}hr_{low} + 0.6× ^{#}hr_{low}) < HR < (0.4× ^{avg}hr_{high} + 0.6× ^{#}hr_{high})
Heart rate variability
HRV < ^{#}hrv_{high}
Respiratory rate
(0.4× ^{avg}rr_{low} + 0.6× ^{#}rr_{low}) < RR < (0.4× ^{avg}rr_{high} + 0.6× ^{#}rr_{high})
Respiratory rate variability
RRV < ^{#}rrv_{high}
Abnormal Lung Sound (ABS) & Cough (CGH)
ABS > 0
CGH > 3
^{#}age defined values from medical research literature.
⁺user input through mobile application interface.
^{avg}Average values for user; must be at least 30 hours of use over more than 7 separate days
```

Digitized acoustic signal in the range of 60 Hz to 2 kHz sampled at least 16 bits is received by the BLE on the smart gateway. The size of the arrival signals may be adjusted according to the memory available on the hardware as well as the intended application or functionality. For example, the computation of RR may require a longer duration as compared to the HR and abnormal heart and lung sound detection as the period of RR is longer. In the face of limited memory space, instead of allocating a larger duration of raw data for RR, pre-processing that may contribute to dimensionality reduction (i.e. through features extraction) may be employed first on the shorter duration of signal and later append these features to meet the optimal duration required for the RR computation. Each of the algorithms employed different signal processing methods. These signal processing methods with focus on signal flow are represented with respective flow diagrams of FIGS. 7 to 9.

Respiratory Rate Estimation

An optimal duration of respiratory sound recordings are processed using the Short-Time-Fourier-Transform (STFT) method 704 as follows::

$$S ( τ , f ) = \int \text{?} \text{?} ( t ) h ( t - τ ) e \text{?} dt \qquad Equation 4$$

$$\text{?} \text{indicates text missing or illegible when filed}$$

where [symbol] is the spectra in time and frequency domains, s(t) is the chest sound (e.g. the digital signal input 702), h(t−τ) is the window function, and τ is the shift in time.

The Fast-Fourier-Transform (FFT) window size and the overlapped window size of STFT are 64 ms and 32 ms, respectively. These values are tuning parameters that may be adjusted through empirical optimisation. To reduce the interference of heart sound, a high pass filter 706 with a cutoff frequency of 100-200 Hz may be applied to the STFT outputs as the heart sounds are predominantly heard at frequency lower than 200 Hz. To accentuate identify the dominant frequency of the spectra, a filter is firstly applied to obtain the averaging spectra, and later the average values are subtracted from the original spectra. This step is also known as peak enhancement 708 while the dominant peaks are accentuated, the small peaks are substituted replaced with a small value of 0.05, as follows in Equation 5:

$${S}_{p} ( t , f ) = \{ \begin{matrix}S ( t , f ) - {S}_{a} ( t , f ) & if S ( t , f ) > {S}_{a} ( t , f ) \\ 0.05 & if S ( t , f ) < {S}_{a} ( t , f )\end{matrix} \qquad Equation 5$$

Assuming there are N dominant frequency components in spectra [symbol] whose peaks values are C₁, C₂, C₃, . . . , C_{N}, a weighted power value of each dominant frequency component may be calculated as follows in Equation 6:

$${p}_{n} = \frac{{C}_{n}}{{\sum }_{n = 1}^{N} {C}_{n}} n = 1 , 2 , 3 , … , N \qquad Equation 6$$

The weighted power values are used for the calculation of entropy 710 as follows in Equation 7:

$${E}_{t} = {\sum }_{n = 1}^{N} {p}_{n} log ( {p}_{n} ) \qquad Equation 7$$

where Eₜ is the entropy computed at each time window.

After all the entropy values (in each FFT time window) have been calculated for 15 seconds, the entropy values are smoothed 712 as follows in Equation 8, as an example:

$${E}_{s} = \frac{1}{M} {\sum }_{t = s}^{t + M - 1} {E}_{t} \qquad Equation 8$$

where Eₛ is the smoothed entropy and M is the size of the smooth filter. M is a tuning parameter. Other smoothing functions (besides that provided by Equation 8) may be applied instead.

The period of the smoothed entropy is calculated using autocorrelation method 714. The time distance between two consecutive peaks of the autocorrelation output is the period of one breath cycle. Respiratory rate is then calculated using the following formula as in Equation 9:

$$RR = \frac{60}{{t}_{b}} \qquad Equation 9$$

where RR is the respiratory rate and t_{b} is the period of one breath cycle or respiratory cycle.

Subsequently, the fuzzy logic 716 examines whether the calculated respiratory rate is bound within the acceptable range of respiratory rate including the upper limit of the abnormal range. If it is within the acceptable range, the results are displayed and updated at the end user, otherwise, the respiratory rate is not updated until the next available reading. A summary of the steps of respiratory rate estimation algorithm is provided in a flow chart 700 depicted in FIG. 7. While the flow chart 700 of FIG. 7 describes a respiratory rate estimation algorithm using entropy space, it should be appreciated and understood that the respiratory rate estimation algorithm is not limited to using entropy space as other types of signal transformation may also be applicable.

Abnormal Respiratory Sound Detection Algorithm

Abnormal respiratory sounds including wheeze, crackles and stridor may be classified determined using this method with different model parameters (if a more model-based classifier is used) or different thresholds (if a simple thresholding method is used). The input signals (e.g. the digital signal input 802) are pre-processed using the STFT method 804. The FFT window size and overlapped window size are 32 ms and 16 ms, respectively. The STFT outputs are high pass filtered 806 with a cutoff frequency of 100-200 Hz to suppress the heart sounds. The peak enhancement method 808 followed by the calculation of entropy calculation 810 and smoothing 812 of entropy may employ the same equation and tuning parameters as the respiratory rate estimation algorithm (see, for example, Equations 7 and 8). It should be appreciated that other smoothing functions may be applied. At least two features may be extracted from the entropy (at 814), for example, entropy difference (see Equation 10) and entropy ratio (see Equation 11). In the simplistic approach, thresholds of entropy difference and entropy ratio are used to classify the abnormal respiratory sounds from the normal respiratory sounds (at 816). The thresholds to may be determined from sets of training data. The thresholding method using two dimensional features may only be chosen if the feature separations of the abnormal and the normal respiratory sounds are able to achieve at least 80% of the total accuracy in the cross-validation. Otherwise, additional features and/or other classification methods may be employed to increase the confidence of the abnormal respiratory rate detection algorithm.

For example,

E_{diff}=Eₘₐₓ−Eₘᵢₙ Equation 10

where E_{diff} is the entropy difference, Eₘₐₓ is the maximum entropy, and Eₘᵢₙ is the minimum entropy.

$${E}_{ratio} = \frac{{E}_{max}}{{E}_{min}} \qquad Equation 11$$

where Eᵣₐₜᵢₒ is the entropy ratio.

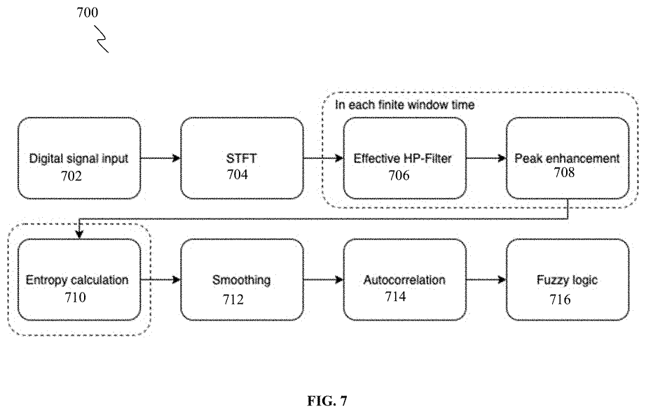

A summary of the steps of the abnormal respiratory sounds detection algorithm is provided in a flow chart 800 depicted in FIG. 8. While the flow chart 800 of FIG. 8 describes an abnormal respiratory sound detection algorithm using entropy space, it should be appreciated and understood that the abnormal respiratory sound detection algorithm is not limited to using entropy space as other types of signal transformation may also be applicable.

Heart Rate Estimation Algorithm

The collected digital data (e.g., the digital signal input 902) may be down sampled at the down sampling sets 904. As an example for illustrative purposes, the down sampling sets 904 may down sample the digital signal input 902 twice instead of once to reduce the size of data while preserving the data resolution. For example, in the first round of down sampling, the total number of samples may be reduced by one-fourth, whereas in the second round of down sampling, the remaining data may be reduced by half. A filter (at 906) may be used to extract heart sounds between 60 Hz and 150 Hz. For example, the filter may be a 49^{th}-order finite impulse response (FIR). The extracted heart sounds may undergo peak enhancement at 908. Autocorrelation method 910 and smoothing method 912 may be used for the estimation of HR. For example, to ensure the autocorrelation 910 captures the periodicity of heart rate, other peak values that are below 15% of the maximum peak are replaced with the value 0. The time difference between two consecutive peaks in the autocorrelation outputs is the period of one cardiac cycle. Heart rate may then be calculated using the following formula in Equation 12:

$$HR = \frac{60}{{t}_{c}} \qquad Equation 12$$

where HR is the heart rate and t_{c} is the period of one cardiac cycle.

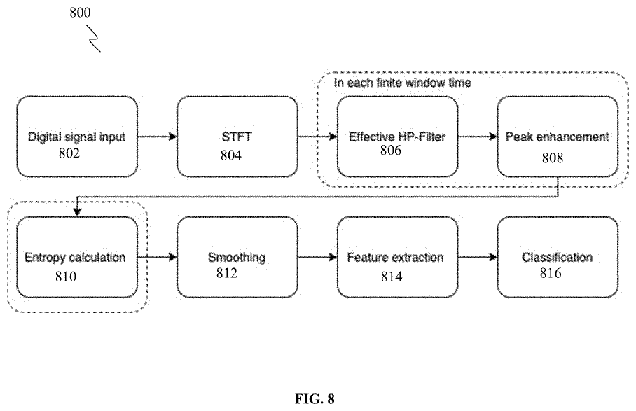

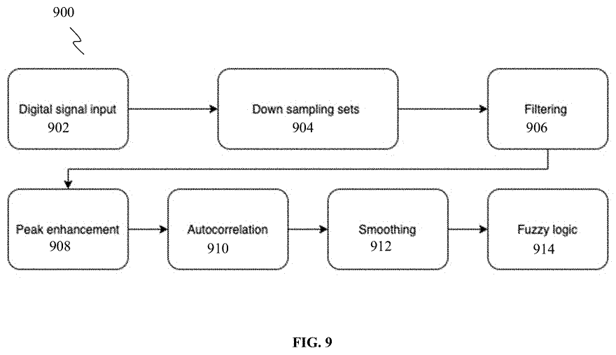

Subsequently, the fuzzy logic 914 examines whether the calculated heart rate is bound within the acceptable range of heart rate. If it is within the acceptable range, the results are displayed and updated to the end user, otherwise, the heart rate is not updated until the next available reading. A summary of the steps of the heart rate detection algorithm is provided in a flow chart 900 depicted in FIG. 9.

User Interfacing Device

The user interfacing device (e.g. 224 of FIG. 2 or 300 of FIG. 3) may be a mobile or web application. Mobile and web app users may subscribe to specific device topics (e.g. app/airesone/v1/AED0001) to receive real-time updates from the gateway device (i.e. the smart gateway 500). Data is received from the gateway device in a 15 seconds interval. The following sets out its general processing method.
A function may be scheduled to run at the end of each day to calculate the average HR and RR for each 15-minute duration. This value is stored in a database and is used for data visualization on the mobile/web app.
The app calls a REST API to execute CRUD functions on the database.
API calls may be handled by API Gateway service. This service validates user's access rights before forwarding the API request to the desired function.

The mobile apps subscribe to specific device topics in order to receive real-time data from the gateway device.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A gateway device operable in a system for monitoring a plurality of bio-signals, the gateway device comprises:
a receiver unit configured to receive data and reconstruct the received data to obtain a digital sound signal representative of the plurality of bio-signals;
a processing unit configured to determine at least one biometric from the digital sound signal and form a digital signal representative of the plurality of bio-signals and the determined at least one biometric, wherein the processing unit is further configured to reconstruct the digital signal into two or more data segments, and wherein each of the two or more data segments comprises a portion of the digital signal, the portion being representative of at least one of:
at least one of the plurality of bio-signals, or
the determined at least one biometric; and
a transmitter unit configured to transmit information including the two or more data segments to an external processing module for further processing.

2. The gateway device as claimed in claim 1, wherein each of the two or more data segments comprises at least three seconds of data.

3. (canceled)

4. The gateway device as claimed in claim 1, wherein the two or more data segments comprise a first segment and a second segment, wherein the first segment has a data length longer than the second segment.

5. The gateway device as claimed in claim 4, wherein the first segment is representative of at least one of:
the at least one of the plurality of bio-signals having an occurrence period between about 3 seconds to about 15 seconds, or
the determined at least one biometric having an occurrence period between about 3 seconds to about 15 seconds; and
the second segment is representative of at least one of:
the at least one of the plurality of bio-signals having an occurrence period between about 3 seconds to about 5 seconds, or
the determined at least one biometric having an occurrence period between about 3 seconds to about 5 seconds.

6. (canceled)

7. (canceled)

8. (canceled)

9. The gateway device as claimed in claim 1, wherein the receiver unit is configured to receive the data via a wireless communication protocol for an optimal communication distance ranging up to about 10 m or a wireless low-energy communication protocol utilizing Low Complexity Communications Codec; and
wherein the transmitter unit is configured to transmit the information to the external processing module in an external server or a cloud-based platform.

10. (canceled)

11. (canceled)

12. The gateway device as claimed in claim 1, further comprising a microphone configured to detect ambient sound surrounding the gateway device, and an analogue-to-digital converter configured to convert the ambient sound to a digital equivalent, wherein the processing unit is further configured to remove noise from the digital sound signal based on the digital equivalent of the ambient sound.

13. A system for monitoring a plurality of bio-signals, the system comprising:
a wearable acoustic device configured to detect analogue sound signals representative of the plurality of bio-signals, and convert the detected analogue sound signals into data representative of the plurality of bio-signals;
a gateway device configured to receive the data from the wearable acoustic device, reconstruct the received data to obtain a digital sound signal, determine at least one biometric from the digital sound signal and form a digital signal representative of the plurality of bio-signals and the determined at least one biometric, reconstruct the digital signal into two or more data segments, and transmit information including the two or more data segments, wherein each of the two or more data segments comprises a portion of the digital signal, the portion being representative of at least one of:
at least one of the plurality of bio-signals, or
the determined at least one biometric; and
a processing module configured to receive the information, and process the received information for visual display and/or audio display to monitor the plurality of bio-signals and/or the determined at least one biometric.

14. The system as claimed in claim 13, further comprising a user interfacing device configured to display the processed information.

15. The system as claimed in claim 13, wherein the wearable acoustic device comprises at least one sensing unit configured to detect the analogue sound signals; a converter configured to convert the detected analogue sound signals into the data; a transmitter configured to transmit the data to the gateway device; and optionally at least one of a conditioning and filtering analogue circuit for removing noise in the analogue sound signals or an amplifier configured to amplify the analogue sound signals prior to conversion into the data, the wearable acoustic device being adapted to reconstruct the data into a form of a plurality of data trenches prior to transmission to the gateway device.

16. (canceled)

17. (canceled)

18. (canceled)

19. The system as claimed in claim 15, wherein each of the plurality of data trenches is a 0.5-second trench.

20. (canceled)

21. A method for monitoring a plurality of bio-signals, the method comprising:
detecting analogue sound signals representative of the plurality of bio-signals;
converting the detected analogue sound signals to data representative of the plurality of bio-signals;
reconstructing the data to obtain a digital sound signal,
determining at least one biometric from the digital sound signal and forming a digital signal representative of the plurality of bio-signals and the determined at least one biometric;
reconstructing the digital signal into two or more data segments, wherein each of the two or more data segments comprises a portion of the digital signal, the portion being representative of at least one of:
at least one of the plurality of bio-signals, or
the determined at least one biometric;
processing information including the two or more data segments for visual display and/or audio display to monitor the plurality of bio-signals and/or the determined at least one biometric.

22. The method as claimed in claim 21, wherein each of the two or more data segments comprises at least three seconds of data.

23. (canceled)

24. The method as claimed in claim 21, wherein the two or more data segments comprise a first segment and a second segment, wherein the first segment has a data length longer than the second segment.

25. The method as claimed in claim 24, wherein the first segment is representative of at least one of:
the at least one of the plurality of bio-signals having an occurrence period between about 3 seconds to about 15 seconds, or
the determined at least one biometric having an occurrence period between about 3 seconds to about 15 seconds; and
the second segment is representative of at least one of:
the at least one of the plurality of bio-signals having an occurrence period between about 3 seconds to about 5 seconds, or
the determined at least one biometric having an occurrence period between about 3 seconds to about 5 seconds.

26. (canceled)

27. The method as claimed in claim 21, wherein the plurality of bio-signals comprises signals derived from bodily sounds, and the determined at least one biometric is selected from the group consisting of a heart rate, a heart rate variability, a respiratory rate, a respiratory rate variability and any combination thereof.

28. (canceled)

29. The method as claimed in claim 21, wherein the steps of reconstructing the data to obtain the digital sound signal, and reconstructing the digital signal into the two or more data segments are to be carried out by a gateway device, the gateway device configured to receive the data via a wireless communication protocol for an optimal communication distance ranging up to about 10 m, or via a wireless low-energy communication protocol utilizing Low Complexity Communications Codec; and
wherein the steps of determining the at least one biometric from the digital sound signal and forming the digital signal representative of the plurality of bio-signals and the determined at least one biometric are carried out by the gateway device.

30. (canceled)

31. (canceled)

32. The method as claimed in claim 29, further comprising reconstructing the data into a form of a plurality of data trenches for transmission to the gateway device.

33. The method as claimed in claim 32, wherein each of the plurality of data trenches is a 0.5-second trench.

34. (canceled)

35. The method as claimed in claim 21, further comprising detecting ambient sound; converting the ambient sound to a digital equivalent; and removing noise from the digital sound signal based on the digital equivalent of the ambient sound, prior to the step of reconstructing the digital signal to obtain the two or more data segments.

36. The method as claimed in claim 21, wherein the step of converting the detected analogue sound signals into the data is based on a sampling rate of at least 1 KHz to obtain the data with resolution of at least 16-bits and above.

37. (canceled)

38. (canceled)
